(19) United States
(12) Patent Application Publication
(10) Pub. No.: US 2007/0299267 A1
(43) Pub. Date: Dec. 27, 2007

(54) PROCESS FOR THE PRODUCTION OF GLYCEROL CARBONATE ESTERS

(76) Inventor: ALFRED WESTFECHTEL, Hilden (DE)

Correspondence Address: COGNIS CORPORATION;PATENT DEPARTMENT, 300 BROOKSIDE AVENUE, AMBLER, PA, 19002, US

(21) Appl. No.: 11/764,973

(22) Filed: Jun. 19, 2007

(30) Foreign Application Priority Data
Jun. 22, 2006 (EP) 06012791

(51) Int. Cl. C08L 67/00; C07D 317/08; C08F 34/02

(52) U.S. Cl. 549/229; 525/27; 526/269

(57) ABSTRACT

The invention relates to a process for the production of glycerol carbonate esters of the formula: 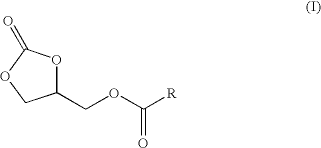
in which R is hydrogen or an alkyl or alkenyl group containing 1 to 3 carbon atoms, which may be linear or branched, wherein, a compound of the formula: 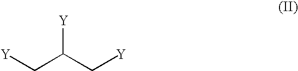
in which Y is a group —O—CO-Z and Z is hydrogen or an alkyl or alkenyl group containing 1 to 3 carbon atoms, which may be linear or branched, is reacted with dimethyl carbonate or diethyl carbonate, to form a compound CH₃—O—CO-Z or CH₃—CH₂—O—CO-Z in the course of the transesterification reaction which is continuously removed from the reaction mixture.

### RELATED APPLICATIONS

This application claims priority from European Patent Application EP06012791.7 filed Jun. 22, 2006, the entire contents of which are incorporated herein by reference.

### FIELD OF THE INVENTION

This invention relates to a process for the production of glycerol carbonate esters.

### BACKGROUND OF THE INVENTION

Esters of glycerol carbonate have been relatively little reported. According to Zephirin Mouloungui et al., Eur. J. Lipid Sci. Technol., 103 (2001) 216-222, the long-chain carbonate esters are obtained by reaction of glycerol carbonate with acid chlorides. In a review of cyclic carbonate-functional polymers in Progress in Organic Coatings, 47 (2003), 77-86, Dean C. Webster presents several synthesis routes for glycerol carbonate methacrylate, including the transesterification of glycerol carbonate with methyl methacrylate. U.S. Pat. No. 3,225,063 describes the production of carbonate esters with cyclic anhydrides in the form of acidic semiesters.

Special publications are devoted to acrylates and methacrylates as unsaturated esters of glycerol carbonate. Thus, U.S. Pat. No. 2,979,514 describes the reaction of acid chlorides and glycerol carbonate. Large excesses of reagents and solvents or entraining agents are used for the reaction. DE 10355830 A1 describes a process in which metal chelate catalysts are used for the transesterification of glycerol carbonate with methyl methacrylate. Here, too, a large excess of acrylate is used.

WO 93/09111 A2 discloses a process for the production of glycerol carbonate esters in which glycerol esters with C₁₋₂₃ fatty acids, more particularly triglycerides based on such fatty acids, are reacted with a carbonate, more particularly dimethyl carbonate or diethyl carbonate, in the presence of a catalyst.

### BRIEF DESCRIPTION OF THE INVENTION

The problem addressed by the present invention was to provide a new process for the production of glycerol carbonate esters.

The present invention relates to a process for the production of glycerol carbonate esters corresponding to formula (I): 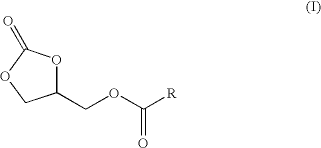
in which R is hydrogen or an alkyl or alkenyl group containing 1 to 3 carbon atoms, which may be linear or branched, characterized in that a compound corresponding to formula (II): 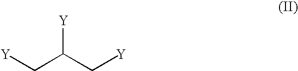
in which Y is a group —O—CO-Z and Z is hydrogen or an alkyl or alkenyl group containing 1 to 3 carbon atoms, which may be linear or branched, is reacted with dimethyl carbonate or diethyl carbonate, the compound CH₃—O—CO-Z or CH₃—CH₂—O—CO-Z formed in the course of the transesterification reaction being continuously removed from the reaction mixture.

The process according to the invention has several advantages. Inexpensive starting materials are used. A methyl ester or ethyl ester of a short-chain carboxylic acid is formed as a secondary product and, besides the actual target product (I), represents a product of value (methyl acetate for example can be used as a solvent). The process according to the invention can be carried out under moderate conditions.

### DETAILED DESCRIPTION OF THE INVENTION

Basically, the choice of the transesterification catalyst is not critical. In principle, any transesterification catalyst may be used. Particularly suitable transesterification catalysts are alcoholates, such as sodium methylate, potassium methylate, or hydroxides, such as sodium hydroxide and potassium hydroxide, and carbonates. The catalysts are preferably used in a quantity of 0.01 to 5% and more particularly 0.5 to 1%, based on the reaction mixture as a whole.

The continuous removal by distillation of the compound CH₃—O—CO-Z formed besides the target compound in the transesterification reaction is a key parameter of the process, ensuring that the reaction can be carried out under moderate conditions and gives light-colored products.

Basically, the reaction temperature is not critical. The reaction is preferably carried out between 40 and 150° C. The temperature range from 60 to 110° C. is particularly preferred.

The reactants are preferably reacted with one another in stoichiometric quantities and, more particularly, in a slight excess of the dimethyl carbonate or diethyl carbonate.

Examples of particularly suitable groups Z in the compounds (II) are hydrogen, methyl, ethyl, n-propyl, i-propyl and —C(CH₃)═CH₂ or —CH═CH₂.

In a preferred embodiment, Z in formula (II) is a methyl group. In another preferred embodiment, Z in formula (II) is a group —C(CH₃)═CH₂ or —CH═CH₂.

If desired, the process according to the invention may be carried out in the presence of a solvent. However, it is preferably carried out in the absence of a solvent.

If desired, the target product (I) may be subjected to purification steps familiar to the expert in order to increase the purity of the product.

The present invention also relates to the use of the compounds (I) as solvents or as high-boiling working liquids. It is of advantage in this regard that the compounds (I) can give off CO₂ through decomposition at elevated temperature (flameproofing effect). In addition, the compounds (I) may be used for the following purposes: hydrophobic emulsifiers, halogen-free lubricant additives, for example for fuels.

### EXAMPLES

### Example 1

### Production of Glycerol Carbonate Acetate (Acetic Acid Ester of Glycerol Carbonate)

The following quantities were used:

| | Ml. weight | Quantity | Quantity | |
|---|---|---|---|---|
| | (g/mol) | (mol) | (g) | |
| Triacetin | 218 | 9 | 1962 | |
| Dimethyl | 90 | 9.9 | 891 | 10% excess |
| carbonate | | | | |
| NaOMe | 54 | 0.26 | 14.25 (47.5 g of a | |
| | | | 30% solution) | |
| HCl (2N) | 36.5 | 0.26 | 134 | |

Procedure:

Triacetin (=triacetic acid ester of glycerol), dimethyl carbonate (DMC) and sodium methanolate (MeONa) were stirred resulting in the formation of a dispersion. The temperature was increased in a nitrogen atmosphere. Distillation began at a bottom temperature of 65 to 70° C. The bottom temperature was slowly increased to 110° C. The quantity of distillate of 1412 g reached the theoretical quantity (1446 g) relatively exactly, the purity of the distillate being monitored by refraction. The bottom product was cooled to 50° C. and neutralized with HCl. 70 g water were added to obtained better separation (salts were dissolved). The aqueous phase was removed. The organic phase was washed out with 360 g water and removed at 50° C. The clear organic phase was dried in a water jet vacuum.

The product was obtained as a clear orange liquid (density=1.34). The yield amounted to 1500 g (85%). The purity (GC analysis) was 98%. The identity of the product was established by ¹H-NMR spectroscopy.

We claim:

1: A process for the production of glycerol carbonate esters of a formula: 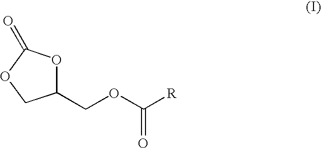
in which R is hydrogen or an alkyl or alkenyl group containing 1 to 3 carbon atoms, which may be linear or branched, which comprises: reacting a compound of formula: 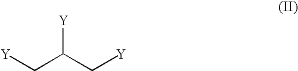
in which Y is a group —O—CO-Z and Z is hydrogen or a linear or branched alkyl or alkenyl group containing 1 to 3 carbon atoms, with dimethyl carbonate or diethyl carbonate, a compound CH₃—O—CO-Z or CH₃—CH₂—O—CO-Z formed in the course of a transesterification reaction being continuously removed from the transesterification reaction mixture.

2: The process of claim 1, wherein, the transesterification reaction is carried out in the presence of a transesterification catalyst.

3: The process of claim 1 wherein the transesterification reaction is carried out at a temperature of from 40° C. to 150° C.

4: The process of claim 1, wherein, Z comprises at least one member selected from the group consisting of hydrogen, methyl, ethyl, n-propyl, i-propyl, —C(CH₃)═CH₂ and —CH₂═CH₂.

5: At least one member selected from the group consisting of solvents, working liquid with a flameproofing effect and lubricant additives for fuels comprising a glycerol carbonate of claim 1.
